(19) United States
(12) Patent Application Publication
(10) Pub. No.: US 2018/0350064 A1
(43) Pub. Date: Dec. 6, 2018

(54) Method And Apparatus For Registering Live Medical Image With Anatomical Model

(71) Applicant: BioProber Corporation, Bellevue, WA (US)

(72) Inventors: Junzheng Man, Bellevue, WA (US); Xuegong Shi, Bellevue, WA (US); Guo Tang, Bellevue, WA (US)

(21) Appl. No.: 15/610,127

(22) Filed: May 31, 2017

(51) Int. Cl. G06T 7/00; G06T 7/30; G06T 7/70; A61B 8/00; A61B 8/08

(52) U.S. Cl. CPC G06T 7/0012; G06T 7/30; G06T 7/70; A61B 8/483; A61B 8/4254; A61B 8/5207; A61B 8/4483

(57) ABSTRACT

Techniques and examples pertaining to registering a medical image of a patient with an anatomical model of the patient are described. A processor of an apparatus may calculate a mapping matrix between a real space and a virtual space using one or more mapping objects. The processor may receive data indicating a physical position of a probe relative to the patient. The processor may subsequently determine a virtual position of the probe in the virtual space based on the mapping matrix such that the virtual position as defined in the virtual space corresponds to the physical position in the real space. The processor may then determine a cut plane of the anatomical model, wherein the medical image may represent an anatomical cross-section of the patient at the cut plane.

### TECHNICAL FIELD

The present disclosure generally relates to medical imaging techniques and, more particularly, to a method and an apparatus for registering live medical images of a patient with an anatomical model of the patient.

### BACKGROUND

Medical imaging involves techniques and processes for creating a visual representation of an interior of a living body, such as a patient. The visual representation, often referred to as a “medical image”, reveals operations or functioning of an organ, a tissue, or a structure of the living body that are not otherwise observable from an exterior of the living body. A medical practitioner, such as a medical doctor or a veterinarian, may refer to the visual representation as part of a medical diagnosis or clinical analysis, and subsequently determine whether or how a medical intervention or treatment may be applied to the living body.

A major challenge of medical imaging resides in a non-intuitive nature of the visual representation, which makes a correct interpretation of the medical image more difficult than it is desired. Typically, it takes a practitioner years of extensive medical training for him or her to be able to interpret or otherwise comprehend a medical image with satisfactory accuracy and detail. In many applications, a medical image constitutes a two-dimensional (2D) cross-section of a body anatomy of a patient, rather than a three-dimensional (3D) replica of the actual body object being examined, be it an organ, tissue or structure. Therefore, it is not trivial to establish a correlation between the 2D medical image and the anatomy of the body object. That is, it is not trivial to identify which anatomical cross-section of the 3D body object the 2D medical image represents.

Therefore, it is desirable to find a more intuitive way to correlate the 2D medical image with the 3D body object being examined.

### SUMMARY

The following summary is illustrative only and is not intended to be limiting in any way. That is, the following summary is provided to introduce concepts, highlights, benefits and advantages of the novel and non-obvious techniques described herein. Select implementations are further described below in the detailed description. Thus, the following summary is not intended to identify essential features of the claimed subject matter, nor is it intended for use in determining the scope of the claimed subject matter.

An objective of the present disclosure is to propose various novel concepts and schemes pertaining to registering a medical image of a patient with an anatomical model of the patient, which can be implemented in various clinical medical or diagnosis applications including ultrasonography scanning. The anatomical model of the patient may be a generic or universal model that is equally applicable to various patients of different genders, heights, weights, ethnicities, ages, and the like, such that patient-specific data may not be required for the registering of the medical image with the anatomical model.

According to an aspect of the present disclosure, an apparatus that registers a medical image of a patient with an anatomical model of the patient is disclosed. The apparatus may include a memory capable of storing data representing the medical image and data representing the anatomical model. The apparatus may also include a processor, wherein the processor may include a space mapping circuit to calculate a mapping matrix between a real space and a virtual space using one or more mapping objects. The real space may be a physical space in which the patient is located, and the virtual space may be a simulation-generated space in which the anatomical model is defined. The processor may also include a physical position tracking circuit to receive data indicating a physical position of a probe relative to the patient, wherein the probe generates the medical image. The processor may also include a virtual position calculation circuit to determine a virtual position of the probe in the virtual space relative to the anatomical model based on the mapping matrix such that the virtual position as defined in the virtual space corresponds to the physical position in the real space. The processor may further include a cut plane calculation circuit to determine a cut plane of the anatomical model based on the virtual position. The medical image may represent an anatomical cross-section of the patient at the cut plane.

In some embodiments, the processor of the apparatus may also include a slice image generation circuit to generate a slice image based on the virtual position of the probe and the anatomical model. The slice image may represent an anatomical cross-section of the anatomical model at the cut plane. The processor may further include a display image rendering circuit to display the medical image overlaid with the slice image. The display image rendering circuit may also display a name of an organ, a tissue, a structure or an anatomical part on either or both of the medical image and the slice image.

According to another aspect of the present disclosure, a method of registering a medical image of a patient with an anatomical model of the patient is disclosed. The method may involve a processor of an apparatus calculating a mapping matrix between a real space and a virtual space using one or more mapping objects. The method may also involve the processor receiving data indicating a physical position of a probe relative to the patient, wherein the medical image is generated by the probe. The method may further involve the processor determining a virtual position of the probe in the virtual space relative to the anatomical model based on the mapping matrix such that the virtual position corresponds to the physical position in the real space. The method may also involve the processor determining a cut plane of the anatomical model based on the virtual position, such that the medical image may represent an anatomical cross-section of the patient at the cut plane.

In some embodiments, the method may involve the processor of the apparatus generating a slice image based on the virtual position of the probe and the anatomical model. The slice image may represent an anatomical cross-section of the anatomical model at the cut plane. The method may also involve the processor rendering the medical image with the slice image.

It is noteworthy that, although description of the proposed scheme and various examples is provided below in the context of ultrasonography scanning for medical purposes, the proposed concepts, schemes and any variation(s)/derivative(s) thereof may be implemented in other non-invasive imaging applications where implementation is suitable. Thus, the scope of the proposed scheme is not limited to the description provided herein.

### BRIEF DESCRIPTION OF THE DRAWINGS

These and other features, aspects, and advantages of the present disclosure will become better understood with regard to the following description, appended claims, and accompanying drawings where:

FIG. 1 is a diagram depicting an example application in accordance with an embodiment of the present disclosure.

FIG. 2 is a diagram depicting relations among an anatomical model, a cut plane of the anatomical model, and a medical image in accordance with an embodiment of the present disclosure.

FIG. 3 is a diagram depicting a data flow in accordance with an embodiment of the present disclosure.

FIG. 4 is a diagram depicting a mapping between a physical space and a simulation-generated space through a mapping matrix in accordance with an embodiment of the present disclosure.

FIG. 5 is a diagram depicting a first kind of mapping objects used for calculating a mapping matrix in accordance with an embodiment of the present disclosure.

FIG. 6 is a diagram depicting a second kind of mapping objects used for calculating a mapping matrix in accordance with an embodiment of the present disclosure.

FIG. 7 is a block diagram depicting an example apparatus in accordance with an embodiment of the present disclosure.

FIG. 8 is a flowchart depicting an example process in accordance with an embodiment of the present disclosure.

### DETAILED DESCRIPTION OF THE PREFERRED EMBODIMENTS

The detailed description of the present disclosure is presented largely in terms of procedures, steps, logic blocks, processing, or other symbolic representations that directly or indirectly resemble the operations of devices or systems contemplated in the present disclosure. These descriptions and representations are typically used by those skilled in the art to most effectively convey the substance of their work to others skilled in the art.

Reference herein to “one embodiment” or “an embodiment” means that a particular feature, structure, or characteristic described in connection with the embodiment can be comprised in at least one embodiment of the present disclosure. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams or the use of sequence numbers representing one or more embodiments of the present disclosure do not inherently indicate any particular order nor imply any limitations in the present disclosure.

To make the above objects, features and advantages of the present disclosure more obvious and easier to understand, the present disclosure is further described in detail below using various embodiments.

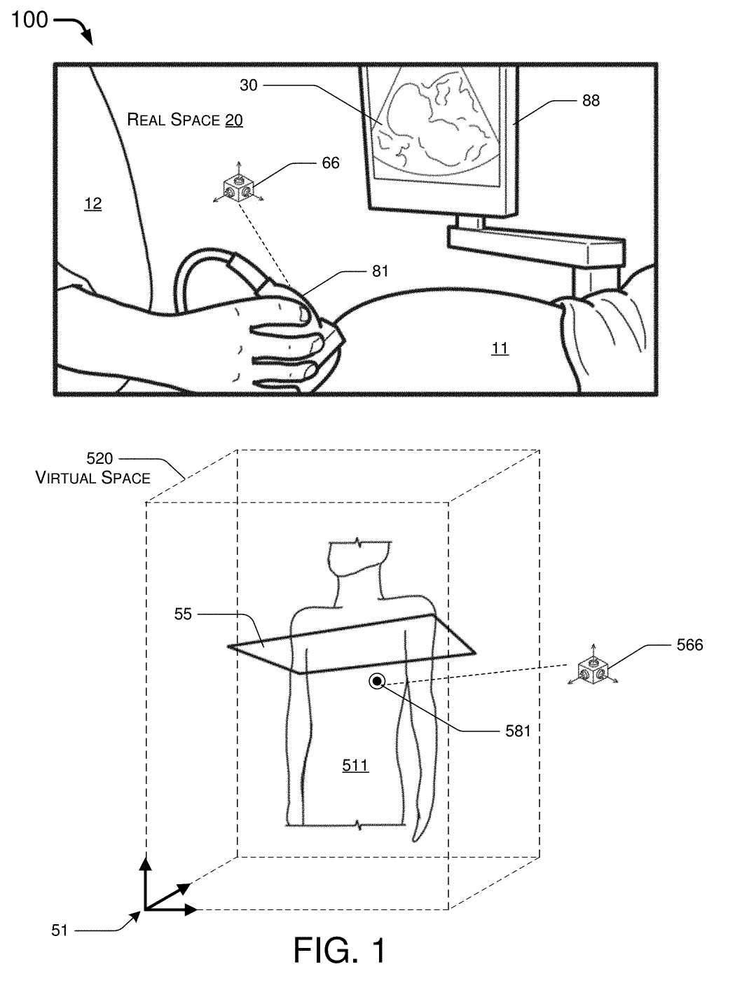

Medical imaging processes and techniques are widely used in contemporary clinical practices to examine or otherwise reveal body operations inside a patient in a non-invasive manner. As mentioned above, a medical practitioner may perform medical diagnosis and clinical analysis using medical imaging techniques, and accordingly determine a medical intervention or treatment to a patient. For example, medical ultrasound, or ultrasonography, is a diagnostic imaging technique based on an application of ultrasound. In general, ultrasound refers to sound waves that are beyond an audible range of frequency to human ears, typically above 20,000 Hz. In a medical application scenario utilizing ultrasound, such as scenario 100 as shown in FIG. 1, short bursts of ultrasound wave may be generated by a hand-held ultrasound transducer (often referred to as a “probe”) 81 that is operated by a practitioner 12. The ultrasound wave may be applied to a patient 11 with probe 81 held close to or touching an external body surface (e.g., abdominal skin) of patient 11. The ultrasound wave would partially penetrate the skin of patient 11 and reach the interior of the patient's body. Various organs, tissues and/or structures inside the body of patient 11 would subsequently echo or otherwise reflect part of the incident ultrasound wave back to probe 81. The reflected ultrasound signal would be picked up, at least partially, by probe 81 and used for constructing or otherwise rendering a visual representation of the internal organs, tissues and/or structures of patient 11, such as an ultrasound image 30 shown in FIG. 1. Ultrasound image 30 may be visually displayed on a monitor or display 88, and may also be digitally recorded or stored in a storage medium such as a computer memory or a hard drive.

An ultrasound image usually constitutes a visual representation of a 2D cross-section of the body anatomy of the patient. For example, ultrasound image 30 of FIG. 1 constitutes a visual representation of a 2D cross-section of the body of patient 11. To explain this idea more clearly, a three-dimensional (3D) human body model 511 of FIG. 1 may be used. Model 511 is an anatomical model of patient 11. As shown in FIG. 1, an imaginary 2D cut plan 55 that intersects with model 511 may identify or otherwise represent a cross-section of patient 11 that is represented by ultrasound image 30, which is generated by probe 81 and shown on display 88. Specifically, cut plan 55 shows where a field of view of probe 81 cuts through a limited area of the patient's body. As shown in FIG. 1, cut plan 55 intersects with model 511 around the chest of model 511, and ultrasound image 30 is a visual representation of an organ, a tissue, a structure 11, or the like, that is internal to the body of patient 11 at cut plane 55.

As presented above, however, it requires a long-term extensive medical training for practitioner 12 to identify in his or her mind what ultrasound image 30 from probe 81 really represents about patient 11. That is, it is neither trivial nor intuitive for practitioner 12 to correlate ultrasound image 30 with cut plane 55 a cross-section of patient at which ultrasound image 30 represents. That posts a major challenge or problem of medical imaging for a practitioner without an extensive medical training to effectively and efficiently identify which anatomical cross-section of a patient a 2D medical image represents. One aspect of the present application is to disclose a method and an apparatus which help or assist practitioner 12 to correctly identify cut plane 55 that is associated with ultrasound image 30. That is, the present application aims to solve this problem by “registering” the medical image with an anatomical model of the patient so that a correlation between the medical image and the anatomy of the body of the patient may be easily established. In the context of the present application, registering A with B means that a spatial relation of A with respect to B is made known. Therefore, if a medical image has been registered with an anatomical model, the cross-section of the anatomical model which the medical image represents is readily identified on the anatomical model. Referring to FIG. 1, an aim of the present application is to register ultrasound image 30 with human body model 511 such that cut plane 55 can be identified.

Typically, probe 81 has a limited field of view. That is, probe 81 is able to sense only a region or area of a limited size, usually in a vicinity of an immediate location of probe 81. As probe 81 moves about the body surface of patient 11, ultrasound image 30 would change accordingly and reveal different parts to the internal of the patient's body. It is also typical that the field of view of the probe is directional. Therefore, even without a translational movement of probe 81 with respect to the patient, the corresponding ultrasound image 30 would change if probe 81 changes its orientation by a rotational movement at a same location. A coordinate system, represented by symbol 66 of FIG. 1, may be used to define, describe or refer to a real space 20 as shown in FIG. 1, a physical space in which patient 11 undergoes the ultrasound process. A movement of probe 81 in real space 20 may then be described or otherwise recorded using the coordinate system of real space 20. The number of coordinates used by the coordinate system typically equals to the degrees of freedom probe 81 can have in real space 20. In scenario 100, probe 81 can have a translational movement as well as a rotational movement in real space 20, having 6 degrees of freedom. The 6 degrees of freedom are illustrated using symbol 66 shown in FIG. 1, which has three mutually orthogonal axes, and each of the axes is associated with a rotation freedom around that respective axis. Namely, a physical position of probe 81 in real space 20 may be uniquely defined using a set of 6 real numbers as its coordinates. For example, 3 of the 6 real numbers may be used to specify a location of probe 81 in real space 20, whereas the rest 3 of the 6 real numbers may be used to specify an orientation of probe 81 in real space 20. In some embodiments, the 3 real numbers specifying the orientation of probe 81 may constitute a normal direction of a primary surface of probe 81 that faces patient 11.

In some embodiments, human body model 511 may be simulation-generated or otherwise manifested in a digital form, which enables a readily determination, through numerical calculation or simulation, of cut plane 55 associated with medical image 30 from probe 81 as long as a spatial relation between probe 81 and patient 11 may also be modeled. As shown in FIG. 1, whereas patient 11 may be modeled by simulation-generated model 511, a spatial relation between the physical position of probe 81 and patient 11 in real space 20 may also be modeled using a simulation-generated virtual space 520. Similar to real space 20 which uses a coordinate system 66, virtual space 520 may employ a coordinate system, represented by symbol 566 of FIG. 1, that also has 6 degrees of freedom. Specifically, model 511 of patient 11 and a virtual position 581 of the physical position of probe 81 may co-exist, by simulation, in virtual space 520, with both referencing to a common origin 51 thereof. Similar to the physical position of probe 81 that uses the 6 coordinates of coordinate system 66 to specify the location and the orientation of probe 81 in real space 20, virtual position 581 may also use 6 coordinates to correspondingly specify in virtual space 520 a location and an orientation, with respect to origin 51, of the corresponding virtual position 581 of probe 81. The 6 coordinates of coordinate system 566 employed by virtual space 520 may have 3 translational degrees of freedom and 3 rotational degrees of freedom, just as coordinate system 66 of real space 20.

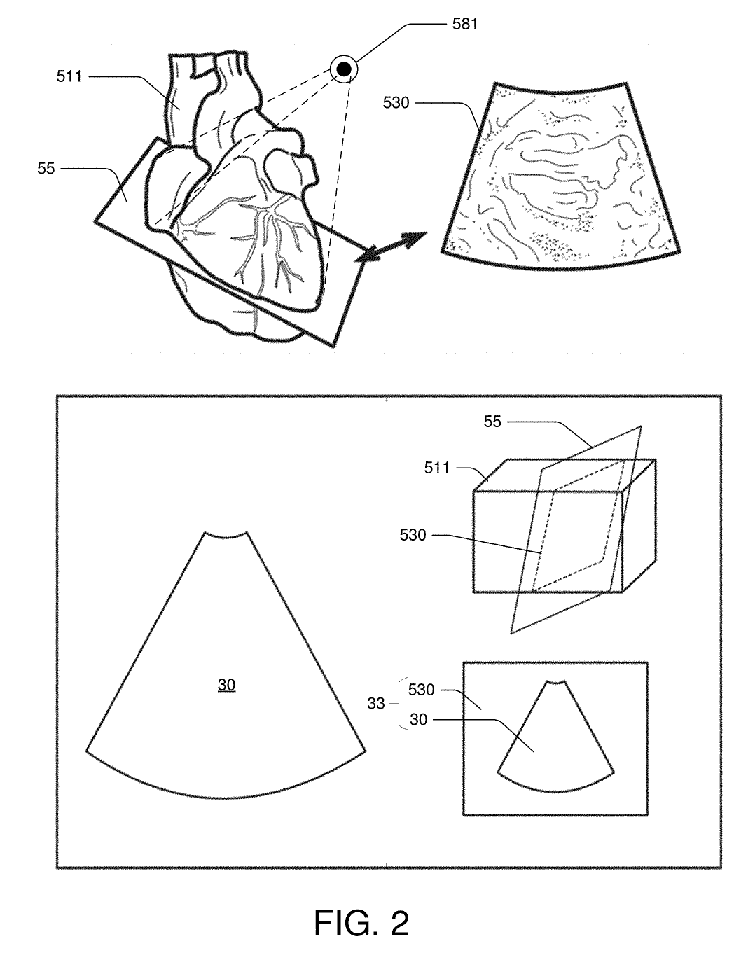

As stated above, since both model 511 and virtual position 581 of probe 81 are simultaneously modeled in a same virtual space (i.e., virtual space 520), a spatial relation of virtual position 581 with respect to model 511 is readily known, and thus the determination of cut plane 55 may be achieved through numerical calculation or simulation. Moreover, a simulated version of ultrasound image 30, often referred to as a “slice image”, may also be achieved based on virtual position 581 and model 511, as well as the spatial relation between the two. The concept is illustrated in FIG. 2 with references to FIG. 1. Firstly, cut plane 55 of anatomical model 511 may be determined based on the spatial relation between virtual position 581 and anatomical model 511. Secondly, the simulated version of ultrasound image 30, or the slice image 530, may be generated or otherwise simulated based on virtual position 581 and model 511. Although slice image 530 may not be completely the same as ultrasound image 30 for various reasons (more details later), they should be quite similar to one another. In some embodiments, 3D model 511 may include names of various anatomical parts it models, such as an organ, a tissue or a structure. Names of at least a few of the various anatomical parts may be included in or otherwise labeled on slice image 530. Slice image 530 may be presented to practitioner 12 side-by-side with the actual ultrasound image 30 to assist a comprehension of the correlation between ultrasound image 30 and model 511. In some embodiments, ultrasound image 30 may be analyzed in view of slice image 530, and various anatomical parts of ultrasound image 30 may thus be identified, and the names of those identified anatomical parts may also be labeled on ultrasound image 30 as they are on slice image 530. In some embodiments, ultrasound image 30 may be rendered with slice image 530. For example, ultrasound image 30 may be overlaid with slice image 530, illustrated as overlaid presentation 33 of FIG. 2, and presented to practitioner 12 through a user interface such as display 88 of FIG. 1. In some embodiments, 3D model 511 along with cut plane 55 identified or otherwise specified on model 511 may also be presented to practitioner 12 to provide further visual aids.

Apparently, every human body is unique. However, it is worth noting that model 511 of FIG. 1 or FIG. 2, though being an anatomical model of patient 11, may be a generic or universal model of a human body, rather than a model specifically tailored to patient 11 or any other particular patient. That is, even without a detailed anatomical model that is specific to a particular patient, the present disclosure enables techniques to register a medical image of the particular patient with a generic anatomical model, such that a medical practitioner is able to readily visualize a spatial relationship between the medical image and the body of the patient, which provides a huge benefit of convenience in practical applications. For example, with anatomical model 511 being a generic human body model and not required to include specific details of patient 11, practitioner 12 of FIG. 1 is still able to visualize a spatial relationship of cut plane 55 of medical image 30 in relation to anatomical model 511 of patient 11, in a real-time fashion, as practitioner 12 moves probe 81 and scans patient 11 with different orientations of probe 81 and/or at different locations in real space 20.

While anatomical model 511 being generic may provide the convenience of not requiring patient-specific details included in anatomical model 511, customization to anatomical model 511 based on certain features related to the particular patient 11 may enhance the accuracy of the registration of medical image 30 with anatomical model 511. For example, anatomical model 511 may be a generic human body model having been customized or otherwise adjusted to capture certain aspects of patient 11 such that a better matching between with anatomical model 511 and patient 11 may be achieved. In some embodiments, anatomical model 511 may include a generic human body model that is customized or adjusted according the gender, height, weight, age, or ethnicity of a particular patient 11. For example, the generic model may be adjusted or stretched longer to fit a patient that is taller than the average. More details regarding adjusting a generic model will be discussed in a later part of the present disclosure.

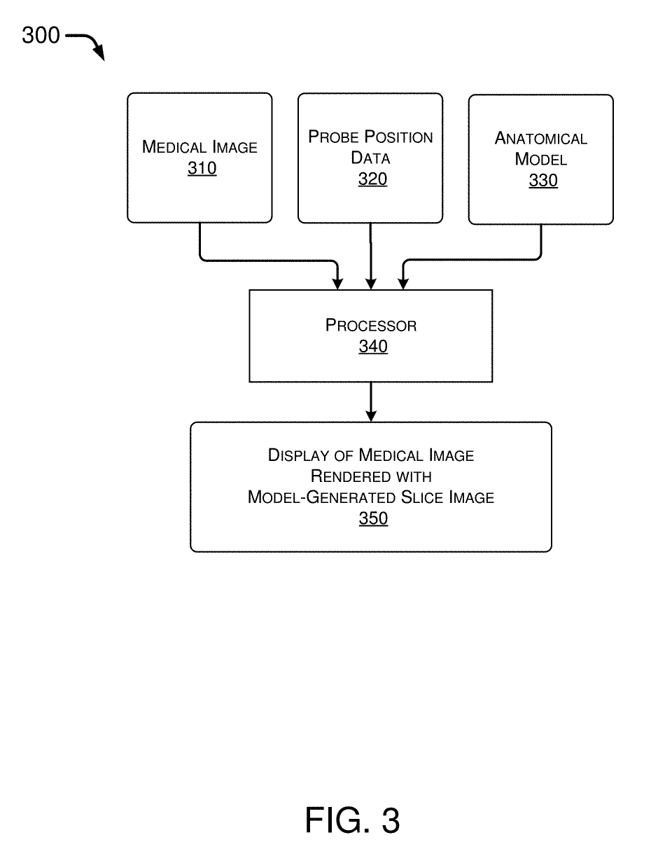

FIG. 3 summarizes a data flow 300 of the process described above regarding registering a medical image of a patient with a digitized 3D anatomical model of the patient such that the medical image may be rendered by a simulated slice image that is generated based on the 3D model. Specifically, processor 340 receives a medical image 310 (such as ultrasound image 30) from a probe (such as probe 81), as well as data 320 indicating a physical position of the probe with respect to the patient. Data 320 may be referred to hereinafter as “probe position data”. In addition, processor 340 receives or otherwise has access to the anatomical model 330. Processor 340 subsequently renders medical image 310 with a slice image (such as slice image 530 of FIG. 2) generated based on anatomical model 330 and probe position data 320, and display the result of the slice-image-rendered medical image.

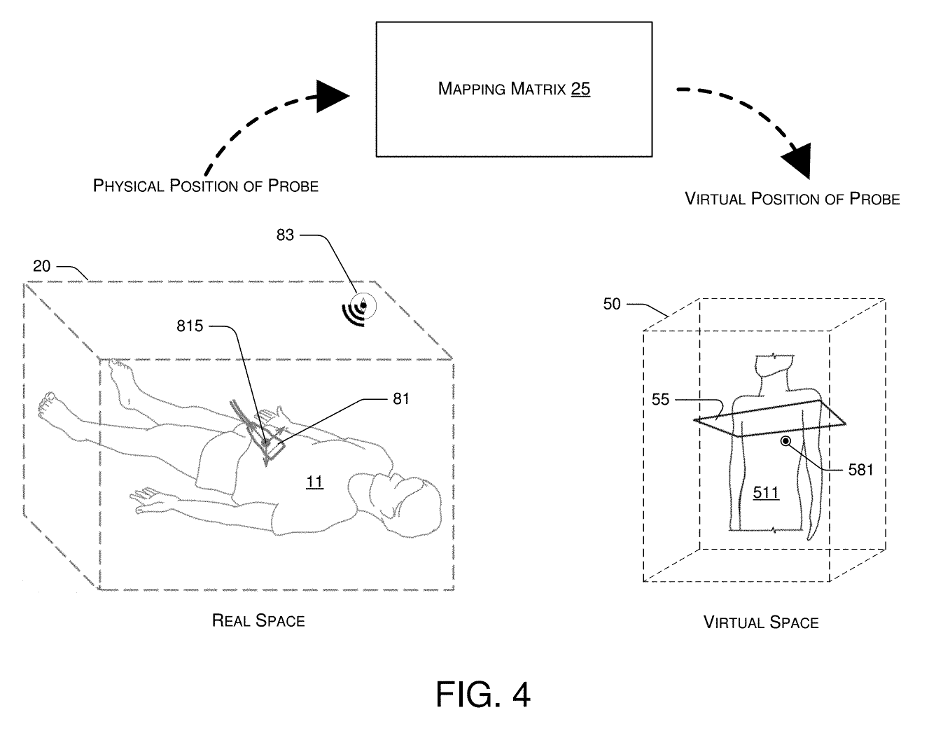

Further details of the process described above are given below along with FIGS. 4-6. In order for probe 81 to provide probe position data 320 of its physical position to processor 340, a navigation transmitter 83 is disposed in real space 20 close to where patient 11 is located, as shown in FIG. 4. Navigation transmitter 83 typically operates with either optical waves or electromagnetic (EM) waves. Using EM waves as an example, navigation transmitter 83 broadcasts the EM waves in real space 20. The EM waves, much like EM signals transmitted by satellites of a global positioning system (GPS), may contain positioning information therein. A EM wave sensor 815 that is embedded in, disposed on or otherwise attached to probe 81 may pick up or otherwise receive some of the EM waves transmitted by navigation transmitter 83. EM wave sensor 815 serves as a position transducer in this context (and maybe referred to as “position transducer” hereinafter), and generates probe position data 320 that indicates the physical position of probe 81 in real space 20 based on the EM waves it receives.

With probe position data 320 provided by position transducer 815 of probe 81, processor 340 of FIG. 3 may determine corresponding virtual position 581 of probe 81 in virtual space 50 relative to anatomical model 511 using a mapping matrix 25 as shown in FIG. 4. As mentioned above, virtual position 581 of probe 81 as defined in virtual space 50 corresponds to the physical position of probe 81 in real space 20. With mapping matrix 25, processor 340 is able to map a physical position relative to patient 11 in real space 20 to a corresponding virtual position relative to anatomical model 511 in virtual space 50. To achieve this purpose, it is also required to establish a mapping between patient 11 in real space 20 and 3D model 511 in virtual space 50, in addition to the mapping between the physical position and the virtual position of probe 81.

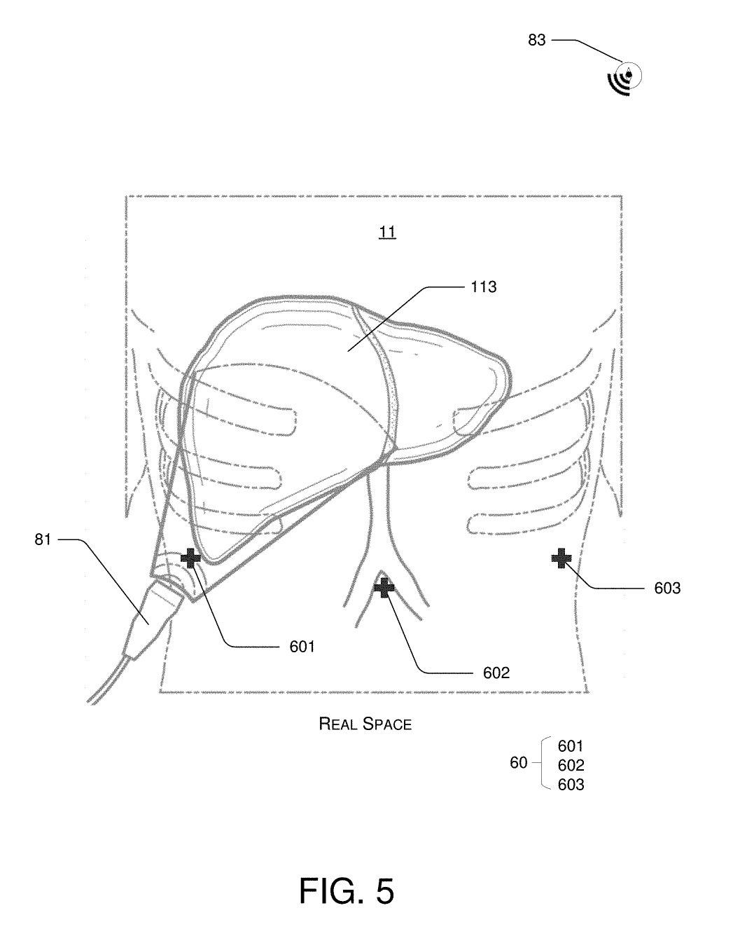

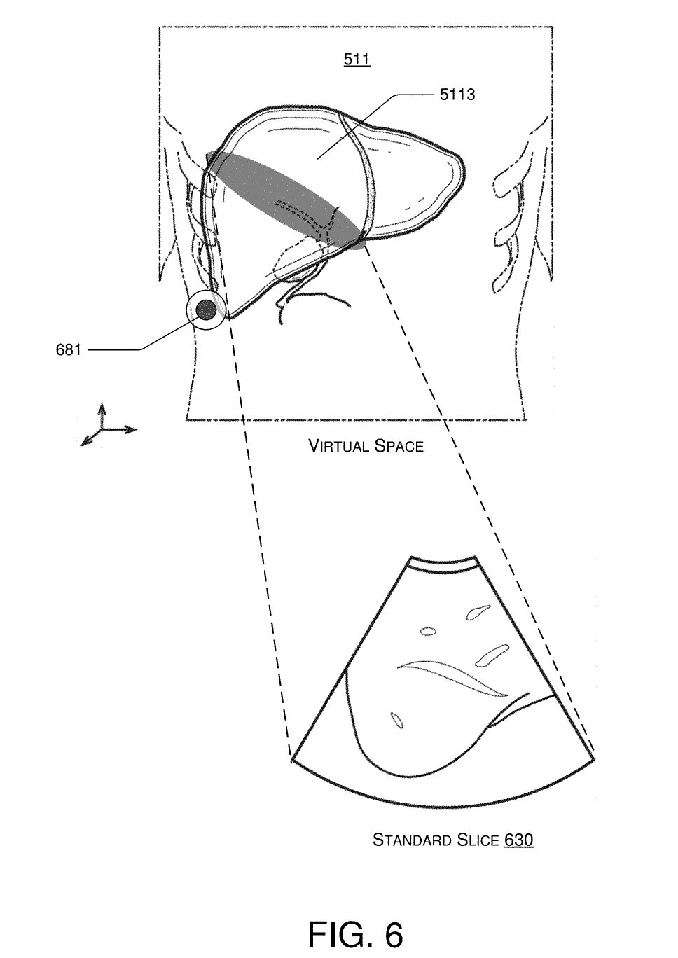

Although mapping matrix 25 is unique for a given pair of real space 20 and virtual space 50, mapping matrix 25 is not given or readily available to processor 340. Instead, it requires processor 340 to go through certain calibration or correlation between real space 20 and virtual space 50 using one or more mapping objects to calculate mapping matrix 25. Mapping matrix 25, as calculated, may yield a satisfactory mapping between real space 20 and virtual space 50, and more specifically, to establish the mapping between patient 11 in real space 20 and 3D model 511 in virtual space 50. Each of FIGS. 5 and 6 demonstrates a respective method of calculating mapping matrix 25 of FIG. 4 through calibration, and each method utilizes one or more mapping objects for the calibration. The calculation of mapping matrix 25 typically involves interpolation of matched positions in real space 20 and virtual space 50 related to the matching objects. Therefore, a higher number of mapping objects involved will generally enhance the performance of matching matrix 25 regarding matching real space 20 and virtual space 50.

FIG. 5 illustrates a method of calculating mapping matrix 25 of FIG. 4 through calibration using matching objects. Specifically, a few predetermined body landmarks, such as body landmarks 60 shown in FIG. 5, are used as the mapping objects. Body landmarks 60 are predetermined locations on the body of patient 11, at which probe 81 may be placed to establish a correlation between real space 20 and virtual space 50. Namely, body landmarks 60 serve as fiducial markers on the body of patient 11 for the mapping between real space 20 and virtual space 50. Typically, the locations of body landmarks 60 are related to a specific body area of patient 11 where the ultrasonography scan is to be performed. For example, for an ultrasonography scan of an area around the liver 113 of patient 11, or any other patient, the locations of body landmarks 60 may be purposefully predetermined at the lower end of sternum of patient 11, the lower end of the rib cage on the left side of the body of patient 11, as well as the lower end of the rib cage on the right side of the body of patient 11, as shown by body landmarks 602, 603 and 601 in FIG. 5, respectively.

When the calibration is performed for the calculation of matching matrix 25, probe 81 is sequentially placed on patient 11 at body landmarks 60 (i.e., body landmarks 601, 602 and 603) of FIG. 5, and corresponding virtual locations (such as a location of virtual position 581 of probe 81 as described in coordinate system 566) of the respective body landmarks 60 are located in virtual space 50 and recorded respectively. That is, probe 81 may firstly be placed at or close to body landmark 601 located at the lower end of the rib cage on the right side of the body of patient 11, and a corresponding location of body landmark 601 in virtual space 50 may be located and recorded. As an example, the three translational coordinates of virtual position 581 of probe 81 may be recorded as the corresponding location of body landmark 601 in virtual space 50. Next, probe 81 may be moved to body landmark 602 located at the lower end of the sternum of patient 11, and the translational coordinates of corresponding virtual position 581 of probe 81 may be recorded as the corresponding location of body landmark 602 in virtual space 50. Finally, probe 81 may subsequently be moved to body landmark 603 located at the lower end of the rib cage on the left side of the body of patient 11, and the translational coordinates of corresponding virtual position 581 of probe 81 may be recorded as the corresponding location of body landmark 603 in virtual space 50. With the corresponding locations of body landmarks 601, 602 and 603 recorded in virtual space 50 by sequentially placing probe 81 on patient 11 at each of body landmarks 601, 602 and 603, processor 340 may calculate mapping matrix 25 accordingly using principles of interpolation.

In general, the more number of the body landmarks 60 are used for the calibration process described in FIG. 5, the more accurate the mapping between real space 20 and virtual space 50 may be achieved through the calculation of mapping matrix 25. While the calculating of mapping matrix 25 may be performed with one or two body landmarks 60 to result in an acceptable mapping accuracy, three or more body landmarks 60 are preferred. A reason for having at least three body landmarks 60 is that the three body landmarks 60 may uniquely determine a cross-section of patient 11 in real space 20. With the three corresponding locations of the three body landmarks 60 located in virtual space 50, a corresponding cut plane of 3D model 511 may also be uniquely determined. With the correlated cross-section of patient 11 and the corresponding cut plane uniquely determined in real space 20 and virtual space 50, respectively, the spatial mapping between real space 20 and virtual space 50 is completed, and mapping matrix 25 of FIG. 4 can be uniquely calculated. As mentioned above, the inherent nature of interpolation used in calculating mapping matrix 25 encourages using a higher number of body landmarks 60, which would enhance the mapping accuracy.

FIG. 6 illustrates another method of calculating mapping matrix 25 of FIG. 4 through calibration using matching objects. In this method, the mapping objects include one or more slice images of anatomical model 511 of patient 11, called “standard slices”. Each of the one or more slice images is associated with a predetermined position in virtual space 50, called “standard position” or “standard probe position”. FIG. 6 shows a standard slice 630 and a standard position 681 associated with standard slice 630. Specifically, standard slice 630 is a slice image of model 511 perceived by probe 81 as probe 81 is placed at a physical position in real space 20 that corresponds to standard position 681 in virtual space 50. Processor 340 of FIG. 3 is able to simulate or otherwise generate standard slice image 630 of anatomical model 511 based on standard position 681.

Typically, a standard position is predetermined based on a specific body area of a patient where the ultrasonography scan is to be performed. For example, for an ultrasonography scan of an area around the liver of patient 11, or any other patient, standard position 681 in virtual space 50 may be purposefully predetermined at the lower end of the rib cage on the right side of anatomical model 511 of patient 11, with an orientation toward a liver 5113 of anatomical model 511, as shown in FIG. 6. Since standard slice 630 is associated with standard position 681, as standard position 681 is purposefully predetermined, standard slice 630 is also considered predetermined.

In general, one standard position (that is, standard position 681) and one standard slice image (that is, standard slice 630) as shown in FIG. 6 are sufficient for the calculation of mapping matrix 25 of FIG. 4. This is because one standard slice image typically has included enough features that, when matched between real space 20 and virtual space 50, would facilitate calculation of a sufficiently accurate mapping matrix 25. Nevertheless, in some embodiments, more standard positions, and thus more standard slice images associated with the standard positions, may be employed to enhance the mapping accuracy of mapping matrix 25.

With standard slice image 630 generated based on standard position 681, probe 81 is moved by practitioner 12 to various positions (i.e., various locations and/or orientations) on the body surface of patient 11 until probe 81 is moved to a so-called “optimal matching position” and an optimal matching is achieved. That is, with probe 81 in the optimal matching position, ultrasound image 30 substantially matches standard slice image 630 to the best ability of practitioner 12 who moves probe 81 around. Since predetermined standard position 681 associated with standard slice image 630 of FIG. 6 is located at the lower end of the rib cage on the right side of anatomical model 511, the optimal matching position associated with standard slice image 630, as decided by practitioner 12, shall be quite close to a physical position in real space 20 that is around body landmark 601 of FIG. 5, which is located at the lower end of the rib cage on the right side of patient 11. Should practitioner 12 move probe 81 to another physical position of real space 20 farther from body landmark 601, such as a physical position near body landmark 602 or body landmark 603, the resulted ultrasound image 30 would not match closely to standard slice image 630, and thus the physical position near body landmark 602 or 603 would not be the optimal matching position for standard slice image 630 associated with standard position 681.

Once the optimal matching position associated with standard position 681 is determined as described above, data indicating the optimal matching position, as detected by navigation transmitter 83 and position transducer 815, may then be sent to processor 340 from probe 81. Processor 340 thereby calculates mapping matrix 25 based on the data indicating the optimal matching position. Similar to the other method of calculating matching matrix 25 using body landmarks 60 of FIG. 5, the more the standard slice images are used for the calibration process described with FIG. 6, the more accurate the mapping between real space 20 and virtual space 50 may be achieved through the calculation of mapping matrix 25. Namely, although the calculating of mapping matrix 25 may be performed with one single standard slice image 630 and the associated optimal matching position and result in an acceptable mapping accuracy, use of more standard splice images is preferred. Due to the inherent nature of interpolation used in calculating mapping matrix 25, a higher number of standard slice image would enhance the mapping accuracy.

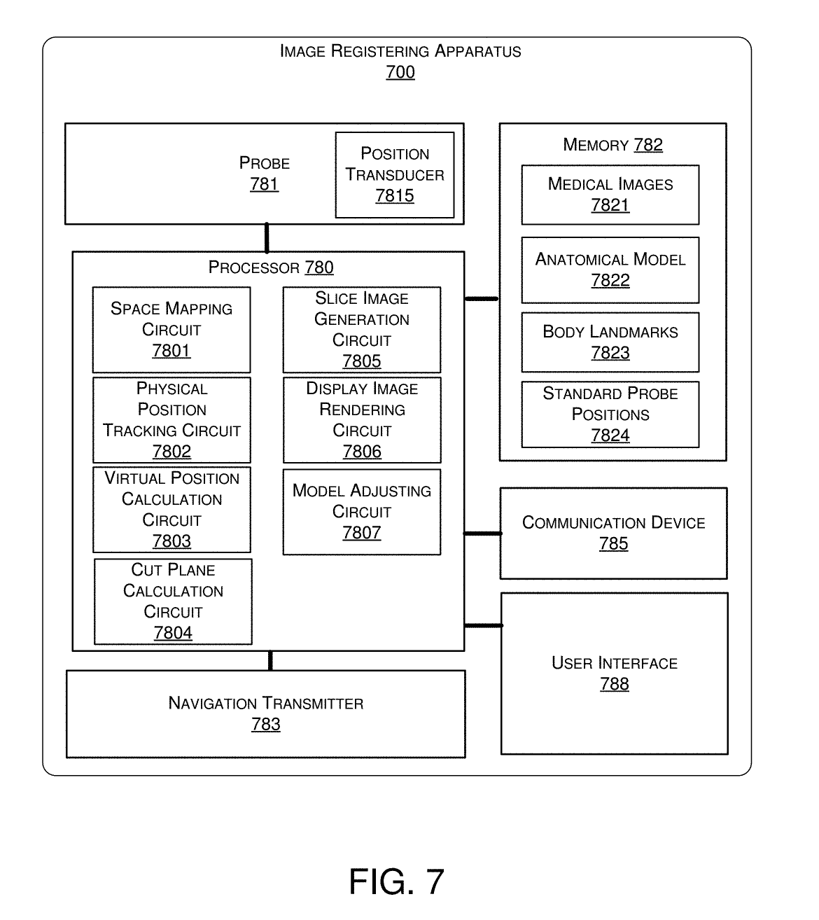

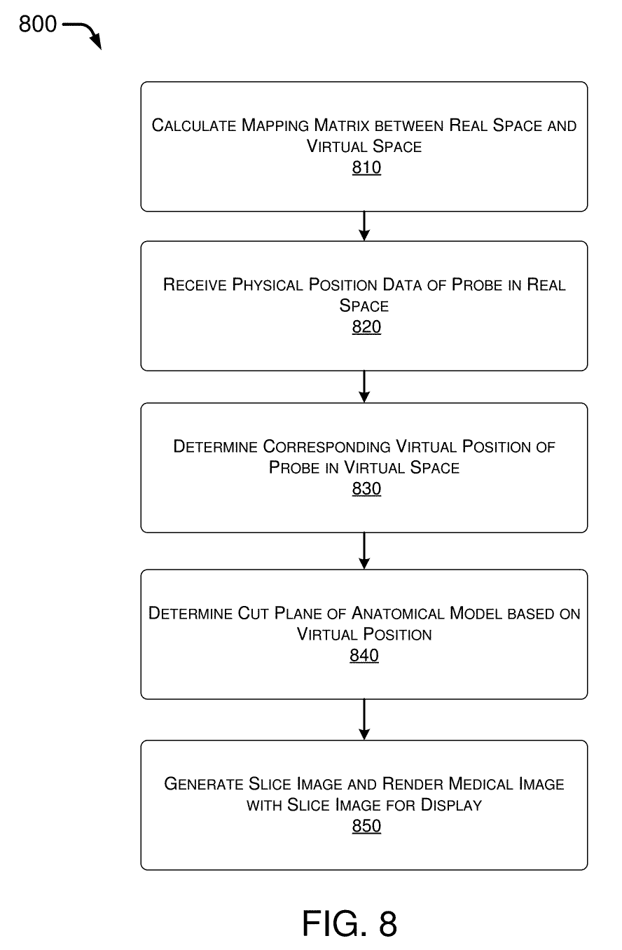

FIG. 7 illustrates an example apparatus, image registering apparatus 700, in accordance with an embodiment of the present disclosure. Apparatus 700 may perform various functions related to techniques, methods and systems described herein, including those described above with FIGS. 1-6, as well as those described below with respect to process 800 of FIG. 8. Apparatus 700 may include at least some of the components of processor 340 of FIG. 3.

Referring to FIG. 7, human health evaluation apparatus 700 may include processor 780 and memory 782. Memory 782 may store data 7821 that represents medical images such as medical image 310 of FIG. 3 or ultrasound image 30 of FIGS. 1 and 2. Memory 782 may also store data 7822 that represents anatomical model 511 of FIGS. 1, 2, 4 and 6, or anatomical model 330 of FIG. 3. Memory 782 may also store data 7823 that represents body landmarks 60 of FIG. 5. Memory 782 may further store data 7824 that represents standard probe positions such as standard probe position 681 of FIG. 6. Memory 782 may be implemented by any suitable technology and may include volatile memory and/or non-volatile memory. For example, memory 782 may include a type of random access memory (RAM) such as dynamic RAM (DRAM), static RAM (SRAM), thyristor RAM (T-RAM) and/or zero-capacitor RAM (Z-RAM). Alternatively or additionally, memory 782 may include a type of read-only memory (ROM) such as mask ROM, programmable ROM (PROM), erasable programmable ROM (EPROM) and/or electrically erasable programmable ROM (EEPROM). Alternatively or additionally, memory 782 may include a type of non-volatile random-access memory (NVRAM) such as flash memory, solid-state memory, ferroelectric RAM (FeRAM), magnetoresistive RAM (MRAM) and/or phase-change memory.

Processor 780 of human health evaluation apparatus 700 may be an embodiment of processor 340 of FIG. 3. In one aspect, processor 780 may be implemented in the form of one or more single-core processors, one or more multi-core processors, or one or more CISC processors. That is, even though a singular term “a processor” is used herein to refer to processor 780, processor 780 may include multiple processors in some implementations and a single processor in other implementations in accordance with the present disclosure. In another aspect, processor 780 may be implemented in the form of hardware (and, optionally, firmware) with electronic components including, for example and without limitation, one or more transistors, one or more diodes, one or more capacitors, one or more resistors, one or more inductors, one or more memristors and/or one or more varactors that are configured and arranged to achieve specific purposes in accordance with the present disclosure. In other words, in at least some implementations, processor 780 is a special-purpose machine specifically designed, arranged and configured to perform specific tasks including registering a medical image with a living body model in accordance with various implementations of the present disclosure.

Processor 780, as a special-purpose machine, may include non-generic and specially-designed hardware circuits that are designed, arranged and configured to perform specific tasks pertaining to registering a medical image of a patient in a physical space with an anatomical model defined in a simulation-generated space in accordance with various implementations of the present disclosure. In one aspect, processor 780 may include a space mapping circuit 7801 that performs specific tasks and functions to calculate a mapping matrix (such as mapping matrix 25 of FIG. 4) between the physical space (such as real space 20) and the simulation-generated space (such as virtual space 50). For instance, space mapping circuit 7801 may be configured to use either body landmarks 60 of FIG. 5 or standard slice image 630 of FIG. 6 as mapping objects to calculate mapping matrix 25 of FIG. 4. In one aspect, processor 780 may include a physical position tracking circuit 7802 that performs specific tasks and functions to receive data indicating a physical position of probe 81 of FIG. 1 in the physical space. For instance, physical position tracking circuit 7802 may be configured to receive data from position transducer 815 embedded in probe 81 of FIG. 4, with the data indicating the physical position of probe 81 relative to patient 11 in real space 20. In one aspect, processor 780 may also include a virtual position calculation circuit 7803 that performs specific tasks and functions to determine a virtual position of probe 81 relative to the anatomical model in virtual space 50. For instance, virtual position calculation circuit 7803 may be configured to determine virtual position 581 of probe 81 relative to anatomical model 511 in virtual space 50 of FIG. 4 based on mapping matrix 25 that is calculated by space mapping circuit 7801. In one aspect, processor 780 may further include a cut plane calculation circuit 7804 that performs specific tasks and functions to determine a cut plane of the anatomical model based on a virtual position, with the medical image representing an anatomical cross-section of the patient at the cut plane. For instance, cut plane calculation circuit 7804 may be configured to determine cut plane 55 of anatomical model 511 of FIG. 4 based on virtual position 581 thereof.

Processor 780, as a special-purpose machine, may include non-generic and specially-designed hardware circuits that are designed, arranged and configured to perform specific tasks pertaining to registering a medical image of a patient in a physical space with an anatomical model defined in a simulation-generated space in accordance with various implementations of the present disclosure. In one aspect, processor 780 may include a slice image generation circuit 7805 that performs specific tasks and functions to generate a slice image based on the virtual position of the probe and the anatomical model. For instance, slice image generation circuit 7805 may be configured to generate slice image 530 of FIG. 2 based on virtual position 581 and anatomical model 511 thereof. Slice image generation circuit 7805 may also be configured to generate standard slice image 630 of FIG. 6 based on predetermined standard position 681 and anatomical model 511 which includes liver 5113. In one aspect, processor 780 may also include a display image rendering circuit 7806 that performs specific tasks and functions to display the medical image overlaid with the slice image generated by slice image generation circuit 7805. Additionally, display image rendering circuit 7806 may also display or generate a 3D image of the anatomical model with the cut plane, as determined by cut plane calculation circuit 7804, identified on the 3D image. Moreover, display image rendering circuit 7806 may further display a name of an organ, a tissue, a structure or an anatomical part on the medical image and/or the slice image. For instance, display image rendering circuit 7806 may be configured to display overlaid presentation 33 having ultrasound image 30 overlaid with slice image 530 as shown in FIG. 2. Alternatively or additionally, display image rendering circuit 7806 may also be configured to display a 3D image of anatomical model 511, with cut plane 55 identified thereon, as shown in FIG. 2.

Processor 780, as a special-purpose machine, may include non-generic and specially-designed hardware circuits that are designed, arranged and configured to perform specific tasks pertaining to registering a medical image of a patient in a physical space with an anatomical model defined in a simulation-generated space in accordance with various implementations of the present disclosure. In one aspect, processor 780 may include a model adjusting circuit 7807 that performs specific tasks and functions to adjust the anatomical model based on some adjustment parameters that are specific to either the patient or a probe that generates the medical image. For instance, model adjusting circuit 7807 may be configured to customize anatomical model 511 of FIGS. 1-4 or anatomical model 330 of FIG. 6 according to some adjustment parameters. The adjustment parameters are further explained below.

In some embodiments, anatomical model 511 or 330 of FIGS. 1-4 and 6 may be a generic model without features or characteristics specific to a particular patient. However, in order to better model a particular patient, in some embodiments, the generic model may be customized using a few adjustment parameters that may reflect certain features or characteristics of the particular patient, and subsequently serve as anatomical model 511 of FIGS. 1-4 or anatomical model 330 of FIG. 6. The adjustment parameters may include characteristics of the particular patient, such as gender, height, weight, age, ethnicity, and the like.

Moreover, probe 81 may have a few probe parameters or operation states that can be set or changed by practitioner 12 to obtain different field of view of ultrasound image 30, even without changing the position (i.e., the location and/or the orientation) of probe 81. Through the probe parameters or operation states, the image depth (i.e., the distance of the cut plane from the probe) may be adjusted. In addition, the zoom factor of the image (i.e., the coverage of the body cross-section represented by the medical image at the cut plane) may also be adjusted for probe 81 in generating ultrasound image 30. Namely, probe parameters and operation states such as image depth and zoom factor are specific to ultrasound image 30 generated by probe 81. To achieve a better correlation between ultrasound image 30 and anatomical model 511, anatomical model 511 may not directly employ a generic model of patient 11. Rather, anatomical model 511 may employ the generic model adjusted or otherwise customized by model adjusting circuit 7807 according to image-specific parameters described above, such as settings of image depth or zoom factor of probe 81.

In some embodiments, apparatus 700 may include a probe 781 (such as probe 81 of FIGS. 1, 4 and 5), movable in the physical space (such as real space 20), as well as a navigator transmitter 783 (such as navigator transmitter 83 of FIG. 4) so as to track movement of probe 781 in the physical space (such as real space 20). Probe 781 may include a position transducer 7815 (such as position transducer 815 of FIG. 4) that is embedded in or otherwise attached to probe 781. In some embodiments, apparatus 700 may also include a communication device 785 capable of wirelessly transmitting and receiving data. For example, communication device 785 may be used by processor 780 to remotely access a data server and receive, transmit or update data 7821, 7822, 7823 and 7824 that may be stored in memory 782. In some embodiments, communication device 785 may also be used by processor 780 to transmit rendered medical images generated by display image rendering circuit 7806 to a display accessible by another medical practitioner located at a remote location for analysis or diagnosis. In some embodiments, apparatus 700 may further include a user interface 788 (such as monitor or display 88 of FIG. 1) for showing the rendered medical images to the patient or a local medical practitioner.

FIG. 8 illustrates an example process 800, in accordance with the present disclosure, for registering a medical image of a patient in a physical space with an anatomical model of the patient defined in a simulation-generated space. Process 800 may include one or more operations, actions, or functions shown as blocks such as 810, 820, 830, 840, and 850. Although illustrated as discrete blocks, various blocks of process 800 may be divided into additional blocks, combined into fewer blocks, or eliminated, depending on the desired implementation. Process 800 may be realized by image registering apparatus 700 of FIG. 7. Process 800 may begin with block 810.

At 810, process 800 may involve a processor (such as processor 340 of FIG. 3 or processor 780 of FIG. 7) calculating a mapping matrix (such as mapping matrix 25 of FIG. 4) between a real space (such as real space 20) and a virtual space (such as virtual space 50) using one or more mapping objects (such as body landmarks 60 of FIG. 5 or standard splice image 630 of FIG. 6). The real space is a physical space in which the patient (such as patient 11 of FIG. 4) is located, whereas the virtual space is a simulation-generated space in which the anatomical model (such as model 511 of patient 11) is defined. Process 800 may proceed from 810 to 820.

At 820, process 800 may involve the processor receiving data indicating a physical position of a probe (such as probe 81 of FIG. 4) relative to the patient. The probe may be an ultrasonography transducer that generates an ultrasonography image of the patient as the medical image. The probe may be provided with a position transducer (such as position transducer 815 of FIG. 4) integrated with the probe. The position transducer may operate by receiving a EM wave transmitted by a navigation transmitter (such as navigation transmitter 83 of FIG. 4) disposed in the real space. Process 800 may proceed from 820 to 830.

At 830, process 800 may involve the processor determining a virtual position (such as virtual position 581 of FIG. 4) of the probe in the virtual space relative to the anatomical model based on the mapping matrix determined in block 810. Specifically, the virtual position as defined in the virtual space corresponds to the physical position of the probe in the real space. Process 800 may proceed from 830 to 840.

At 840, process 800 may involve the processor determining a cut plane (such as cut plane 55 of FIG. 4) of the anatomical model based on the virtual position determined in block 830. The medical image represents an anatomical cross-section of the patient at the cut plane. Process 800 may proceed from 840 to 850.

At 850, process 800 may involve the processor generating a slice image (such as slice image 530 of FIG. 2) based on the virtual position of the probe, as determined in block 830, and the anatomical model. The slice image may represent an anatomical cross-section of the anatomical model at the cut plane as determined in block 840. At 850, process 800 may also involve the processor rendering the medical image with the slice image for display. In some embodiments, the rendering of the medical image with the slice image may involve the processor overlaying the slice image with the medical image to generate an overlaid presentation (such as overlaid presentation 33 of FIG. 2). The overlaid presentation may be shown by the processor on a display device (such as display 88 of FIG. 1). In some embodiments, the rendering of the medical image with the slice image may involve the processor presenting the splice image side-by-side with the medial image. In some embodiments, the rendering of the medical image with the slice image may involve the processor generating a 3D image of the anatomical model with the cut plane identified on the 3D image. The 3D image along with the cut plane identified on the 3D image may be shown by the processor on a display device (such as display 88 of FIG. 1). In some embodiments, the rendering of the medical image with the slice image may also involve the processor labeling a name of an organ, a tissue, a structure, an anatomical part or a body landmark on the medical image and/or the slice image.

The present disclosure has been described in sufficient details with a certain degree of particularity. It is understood to those skilled in the art that the present disclosure of embodiments has been made by way of examples only and that numerous changes in the arrangement and combination of parts may be resorted without departing from the spirit and scope of the present disclosure as claimed. Accordingly, the scope of the present disclosure is defined by the appended claims rather than the foregoing description of embodiments.

### Additional Notes

The herein-described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

Further, with respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

Moreover, it will be understood by those skilled in the art that, in general, terms used herein, and especially in the appended claims, e.g., bodies of the appended claims, are generally intended as “open” terms, e.g., the term “including” should be interpreted as “including but not limited to,” the term “having” should be interpreted as “having at least,” the term “includes” should be interpreted as “includes but is not limited to,” etc. It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to implementations containing only one such recitation, even when the same claim includes the introductory phrases “one or more” or “at least one” and indefinite articles such as “a” or “an,” e.g., “a” and/or “an” should be interpreted to mean “at least one” or “one or more;” the same holds true for the use of definite articles used to introduce claim recitations. In addition, even if a specific number of an introduced claim recitation is explicitly recited, those skilled in the art will recognize that such recitation should be interpreted to mean at least the recited number, e.g., the bare recitation of “two recitations,” without other modifiers, means at least two recitations, or two or more recitations. Furthermore, in those instances where a convention analogous to “at least one of A, B, and C, etc.” is used, in general such a construction is intended in the sense one having skill in the art would understand the convention, e.g., “a system having at least one of A, B, and C” would include but not be limited to systems that have A alone, B alone, C alone, A and B together, A and C together, B and C together, and/or A, B, and C together, etc. In those instances where a convention analogous to “at least one of A, B, or C, etc.” is used, in general such a construction is intended in the sense one having skill in the art would understand the convention, e.g., “a system having at least one of A, B, or C” would include but not be limited to systems that have A alone, B alone, C alone, A and B together, A and C together, B and C together, and/or A, B, and C together, etc. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

From the foregoing, it will be appreciated that various implementations of the present disclosure have been described herein for purposes of illustration, and that various modifications may be made without departing from the scope and spirit of the present disclosure. Accordingly, the various implementations disclosed herein are not intended to be limiting, with the true scope and spirit being indicated by the following claims.

What is claimed is:

1. A method of registering a medical image of a patient with an anatomical model of the patient, comprising:
calculating, by a processor, a mapping matrix between a real space and a virtual space using one or more mapping objects, the real space being a physical space in which the patient is located, the virtual space being a simulation-generated space in which the anatomical model is defined;
receiving, by the processor, data indicating a physical position of a probe relative to the patient, the probe generating the medical image, the physical position being defined in the real space;
determining, by the processor, a virtual position of the probe in the virtual space relative to the anatomical model based on the mapping matrix such that the virtual position as defined in the virtual space corresponds to the physical position in the real space; and
determining, by the processor, a cut plane of the anatomical model based on the virtual position, the medical image representing an anatomical cross-section of the patient at the cut plane.

2. The method of claim 1, wherein:
the probe comprises an ultrasonography transducer,
the medical image comprises a two-dimensional (2D) ultrasonography cross-section, and
the anatomical model comprises a three-dimensional (3D) digital model of an organ, a tissue, a structure or an anatomical part.

3. The method of claim 1, wherein the probe is provided with a position transducer integrated with the probe to generate and track the physical position of the probe.

4. The method of claim 1, wherein:
the one or more mapping objects comprise three or more predetermined body landmarks of the patient,
the calculating of the mapping matrix comprises sequentially locating each of the three or more body landmarks in the virtual space with the probe sequentially placed on the patient at each of the three or more body landmarks in the real space.

5. The method of claim 1, wherein:
the one or more mapping objects comprise one or more predetermined slice images of the anatomical model each generated by the processor based on a respective predetermined position of the probe in the virtual space, and
the calculating of the mapping matrix comprises, for a respective one of the one or more predetermined slice images, receiving data indicating a corresponding physical position of the probe at which the probe generates a respective medical image of the patient that substantially matches the respective one of the one or more predetermined slice images.

6. The method of claim 1, wherein:
the probe is movable in the real space both translationally and rotationally,
the physical position comprises a location and an orientation of the probe in the real space, and
the virtual position comprises a location and an orientation of the probe in the virtual space.

7. The method of claim 1, further comprising:
generating, by the processor, a three-dimensional (3D) image of the anatomical model with the cut plane identified on the 3D image.

8. The method of claim 1, further comprising:
generating, by the processor, a slice image based on the virtual position of the probe and the anatomical model, the slice image representing an anatomical cross-section of the anatomical model at the cut plane; and
rendering, by the processor, the medical image with the slice image.

9. The method of claim 8, wherein the rendering of the medical image with the slice image comprises overlaying the slice image with the medical image.

10. The method of claim 8, further comprising:
labeling a name of an organ, a tissue, a structure or an anatomical part on one or both of the medical image and the slice image based on the anatomical model.

11. The method of claim 1, wherein the anatomical model comprises a generic model customized with one or more adjustment parameters specific to the patient, and wherein the one or more adjustment parameters comprise one or more of a gender, a height, a weight, an ethnicity, and an age of the patient.

12. The method of claim 1, wherein the anatomical model comprises a generic model customized with one or more adjustment parameters specific to the medical image, and wherein the one or more adjustment parameters comprise one or more of an image depth of the medical image and a zoom factor of the medical image.

13. An apparatus that registers a medical image of a patient with an anatomical model of the patient, comprising:
a memory capable of storing data representing the medical image and data representing the anatomical model; and
a processor, comprising:
a space mapping circuit to calculate a mapping matrix between a real space and a virtual space using one or more mapping objects, the real space being a physical space in which the patient is located, the virtual space being a simulation-generated space in which the anatomical model is defined;
a physical position tracking circuit to receive data indicating a physical position of a probe relative to the patient, the probe generating the medical image, the physical position being defined in the real space;
a virtual position calculation circuit to determine a virtual position of the probe in the virtual space relative to the anatomical model based on the mapping matrix such that the virtual position as defined in the virtual space corresponds to the physical position in the real space; and
a cut plane calculation circuit to determine a cut plane of the anatomical model based on the virtual position, the medical image representing an anatomical cross-section of the patient at the cut plane.

14. The apparatus of claim 13, wherein the processor further comprises:
a display image rendering circuit to generate a three-dimensional (3D) image of the anatomical model with the cut plane identified on the 3D image, display the 3D image with the cut plane identified on the 3D image, and display a name of an organ, a tissue, a structure or an anatomical part on the 3D image.

15. The apparatus of claim 13, wherein the operations further comprise:
a slice image generation circuit to generate a slice image based on the virtual position of the probe and the anatomical model, the slice image representing an anatomical cross-section of the anatomical model at the cut plane;
a display image rendering circuit to display the medical image overlaid with the slice image and display a name of an organ, a tissue, a structure or an anatomical part on one or both of the medical image and the slice image.

16. The apparatus of claim 15, further comprising:
a user interface to display the medical image overlaid with the slice image; and
a communication device to receive or transmit the data representing the medical image, the data representing the anatomical model, data representing the slice image, or a combination thereof.

17. The apparatus of claim 13, further comprising:
a navigation transmitter disposed in a vicinity of the patient to transmit an optical or electromagnetic signal received by the probe; and
the probe, wherein the probe is provided with a position transducer integrated with the probe to generate and track the physical position of the probe based on the optical or electromagnetic signal.

18. The apparatus of claim 13, wherein:
the one or more mapping objects comprise three or more predetermined body landmarks of the patient,
the space mapping circuit calculates the mapping matrix by sequentially locating each of the three or more body landmarks in the virtual space with the probe sequentially placed on the patient at each of the three or more body landmarks in the real space.

19. The apparatus of claim 13, wherein:
the one or more mapping objects comprise one or more predetermined slice images of the anatomical model each generated by the processor based on a respective predetermined position of the probe in the virtual space, and
the space mapping circuit calculates the mapping matrix by, for a respective one of the one or more predetermined slice images, receiving data indicating a corresponding physical position of the probe at which the probe generates a respective medical image of the patient that substantially matches the respective one of the one or more predetermined slice images.

20. The apparatus of claim 13, wherein:
the probe is movable in the real space both translationally and rotationally,
the physical position comprises a location and an orientation of the probe in the real space, and
the virtual position comprises a location and an orientation of the probe in the virtual space.
